(19) United States
(12) Patent Application Publication
(10) Pub. No.: US 2019/0259593 A1
(43) Pub. Date: Aug. 22, 2019

(54) A METHOD FOR THE DETECTION AND CHEMICAL SPECIATION OF ORGANIC RADICALS IN NATURAL AND ARTIFICIAL GAS MIXTURES

(71) Applicant: WEGMANN MARIN, Tyresö (SE)

(72) Inventor: Barbara NOZIERE, Tyresö (SE)

(73) Assignee: WEGMANN MARIN, Tyresö (SE)

(21) Appl. No.: 16/327,812

(22) PCT Filed: Sep. 20, 2017
(86) PCT No.: PCT/SE2017/050914

§ 371 Date: Feb. 23, 2019

(30) Foreign Application Priority Data
Sep. 23, 2016 (SE) 1600267-7

(51) Int. Cl. H01J 49/00; H01J 49/26; H01J 49/24; H01J 49/04; G01N 27/62; G01N 33/00

(52) U.S. Cl. CPC H01J 49/0072; H01J 49/26; G01N 33/0016; H01J 49/04; G01N 27/62; H01J 49/24

(57) ABSTRACT

The present invention is related to a method to detect and speciate short-lived chemical substances such as organic radicals in natural and artificial gaseous environments.

In short, positive charged reagent ions (AH+) are created in a soft ionization unit (9) and then accelerated into a so called drift chamber (2), where they collide with injected radical molecules (RO₂), after which the newly born product ions (RO₂H⁺+A) are detected and spectated in a sensitive mass spectrometer (4) according to their masses.

The main field of application of the invention would be the detection of gas-phase organic radicals in ambient air, for research activities on air chemistry, air quality, and medical investigation of the impact of air pollution on human health, as well as for routine monitoring activities in the same fields of indoor and outdoor environment monitoring, health monitoring.

The present invention is related to a method to detect and speciate short-lived chemical substances such as organic radicals in natural and artificial gaseous environments. “Speciate” means in the present document that different compounds or radicals are distinguished from their molecular weight.

Organic radicals are essential compounds present in both natural and polluted environments, natural and industrial combustion processes, biological systems including the human body, and many other chemical systems.

Organic radicals such as but not limited to, peroxy radicals “RO₂” where R is an organic group: CH₃O₂, C₂H₅O₂, C₃H₇O₂ . . . ), alkoxyl radicals (“RO”, CH₃O . . . ) and Criegee intermediates (R—HO₂: CH₂O₂ . . . ), are important but instable, short-lived chemical compounds produced in numerous gas and liquid chemical systems such as ambient air, natural waters, and other natural environments, natural and industrial combustion mixtures (including engines) and biological systems. They play essential roles in the mechanisms and output of all these systems such as ozone formation in the atmosphere, “knock” and auto-ignition in engines, and oxidative stress, cancer, cardiovascular diseases and also in Alzheimer disease etc, in medicine.

But, because of their instability and small concentrations, the observation of such radicals involves many challenges. In particular, few techniques can differentiate between different radicals, for instance between peroxy radicals carrying different organic groups, and, generally between organic radicals with different molecular weight, such as CH₃O₂ and C₂H₅O . This is a major challenge for the development of all these fields of chemistry because radicals with different organic groups have a very different reactivity. Having a technique able to differentiate between radicals of different molecular weight (“speciated measurement”) would thus be a major progress in all the disciplines mentioned above.

Until now, the existing techniques to measure these radicals belong to two groups: either they can be applied to complex systems, such as natural environments, but can not speciate the radicals, or they can speciate the radicals but can only be applied to simplified systems in laboratory. Examples of techniques from the first group, i.e. applicable to complex systems, are the classical techniques such as UV absorption spectroscopy, Electron Spin Resonance and Electron Paramagnetic Resonance spectroscopies (EPR), EPS and ESR being particularly used in biochemistry and medicine. All these techniques produce identical spectra for radicals with different organic groups, thus making impossible to differentiate between them. Other techniques belonging to this group are those developed to detect organic radicals in ambient air, All of them are based on chemical conversion or “chemical amplification”, which consists in reacting all the different radicals with a single reagent, producing the same product, which is detected. For instance, the most common reaction is that of RO₂s with NO to produce NO₂,

RO₂+NO->NO₂+HO₂+products,

where only NO₂ is measured by luminescence (“PERCA”), mass spectrometry, or other techniques. Since the same product is monitored, for instance NO₂, regardless of the radical, this technique excludes the speciation of radicals. Variations of this technique consist in monitoring HO₂ radicals instead of NO₂ in the above reaction, by Laser Induced Fluorescence (ROxLIF, and FAGE), or to react the RO₂s with isotopically labelled ³⁴SO₂ to produce isotopically labelled sulfuric add (H₂³⁴SO₄), which is measured by mass spectrometry (ROxMAx or PerCIMS). In the best case, semi-speciated measurements of RO₂s have been achieved with PAGE, by distinguishing the sum of the unsaturated radicals from the sum of the aromatic ones. But this level of distinction could not be improved and does not allow to distinguish between specific radicals.

Among the second group of techniques, those able to speciate organic radicals but that are limited to simple laboratory applications so far, mass spectrometry is the main one, especially when coupled with soft ionization techniques such as Chemical Ionization. In chemical ionization mass spectrometry, each compound or radical analyzed produces a specific ion, of mass directly related to the mass of the parent compound, thus different organic radicals can be distinguished by the mass of their ion products (mass-based speciation). Numerous chemical ionization techniques have thus been explored for the detection of organic radicals, such as electron transfer, with SF6− and O₂− for instance, which works well for Criegees intermediates and RO₂S, and proton transfer with H₃O+ and clusters H₃O+(H₂O)n, which works well with RO₂S (Scholtens et al., 1999; Hanson et al., 2004).

These techniques are however only applicable to low-pressure chemical systems (for instance SF and negative ionization), or have been directly integrated to specific laboratory set-ups until now, excluding their application to atmospheric or high-pressure systems and to complex systems, such as ambient air or combustion mixtures.

Proton transfer mass spectrometry is widely used for the detection of stable organic gases in complex systems such as ambient air, indoor air, exhaust from industrial processes (food industry . . . ) and even human breath (Lindinger at al., 1998). But these instruments, in particular those commercialized by Ionicon Analytik, Gmbh do not detect RO₂s or other organic radicals.

Thus, in spite of many decades of efforts in the scientific community, no technique able to detect and speciate organic radicals in high-pressure and complex gas systems, is yet available.

The present invention solves this situation after a 15 years research effort to detect and speciate organic radicals in the atmosphere. After some initial studies starting in 2002, which in 2004-2005 resulted in an idea how to solve this problem, whereupon the first functioning analysis instrument was finished a couple of years later and the first radicals in complex gas mixtures could be detected in 2010.

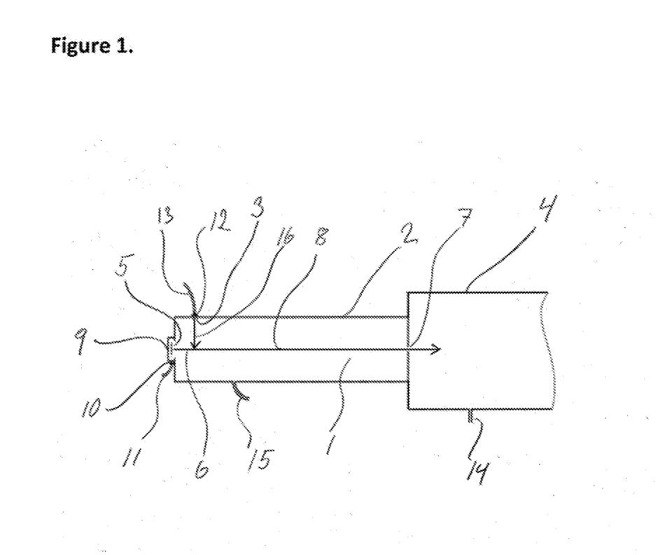

The present invention solves the above mentioned problems, by having the features defined in the independent claim. An embodiment of the invention is shown in FIG. 1.

### TECHNICAL DESCRIPTION

The invention is based on combining different methods and techniques, available in the field of analytical chemistry but never combined and operated according to the invention, to be able to achieve the speciated detection of organic radicals (i.e. differentiate between organic radicals of different molecular weight) in near-atmospheric pressure and complex gas systems. These methods and techniques are:)
a) using proton-transfer ionization to ionize the radicals to be detected. This consists in reacting the radicals with reagent ions of the type “AH⁺” in a so-called drift chamber, where the reagent ions exchange a proton with the radicals: RO₂+AH+→RO₂H⁺+A,
b) operating the drift chamber (thus the reaction between reagent ions and radicals) at intermediate pressure (typically 5-200 mbar, but possibly up to 500 mbar),
c) applying a low electric field to the drift chamber, typically less than 100 Td. (1 Td=1 W cm⁻² at around 3 mbar, which reflects the collision energy between the reagent ions and the radicals),.
d) sampling the radicals from the gas mixtures where they are present by external sampling into the drift chamber. This means that the sampling of the radicals is physically separated from the drift chamber and that the operating conditions of the drift chamber can be chosen and optimized independently from the conditions of the gas mixture in which the radicals are present and
e) using a high-sensitivity mass spectrometer (detection limit <100 pptV (eg. a higher sensitivity gives a lower detection limit) to select and detect the ionized radicals.

Counter-examples of external sampling are all instrumental set-ups where the drift region is also entirely or partly used as sampling region. This include set-ups where the ionization of the radicals is taking place directly in the same reactor as they are created (Scholtens et al., 1999) and those where a large part of the drift region is used as sampling port, as in Hanson et al. 2004, where the reactor was “connected to the drift region by a large port” . . . “acting both as a sampling port and as a drift region”. In these cases, the operating conditions of the drift region, in particular the pressure, could not be varied independently from the conditions in the gas mixture where the radicals are presents and the sampling flow is not actively controlled (typically a passive control, such as a critical orifice or pinhole between both region imposes a constant pressure difference between them). “External sampling” includes the use of active means to control the sampling flow and the drift region pressure, such as equipping the sampling inlet with a valve or a flow controller.

In short, positive charged reagent ions (AH+) are created in a soft ionization unit and then accelerated into a so called drift chamber, where they collide with perpendicularly injected radical molecules (RO₂), after which the newly born product ions (RO₂H⁺+A) are detected and speciated in a sensitive mass spectrometer.

The different parts, necessary for the invention are aligned along a centerline, namely 1) a drift chamber designed for proton transfer ionization at intermediate pressure (typically 5-200, but possibly up to 500 mbar) 2) an external sampling unit optimized for the detection of radicals, and 3) a sensitive mass spectrometer on which the drift region is mounted.

The drift chamber, preferably symmetrically placed around the centerline is pressure tight and under vacuum, typically 5-200 mbar, but possibly up to 500 mbar, and has in one end an inlet opening for entering reagent ions and in the other end an exit pin hole for exiting product ions entering into the mass spectrometer. Preferably the opening and pinhole are situated along the centerline. The drift chamber can be made of a range of materials, such as, but not limited to stainless steel, glass, Teflon, Delrin, etc, and is typically between 5 and 10 cm in length, preferably less than 30 cm, and 1 and 10 cm in diameter.

The length of the drift chamber is chosen to ensure two conditions:

1) that the reaction time between the reagent ions and the analytes (in particular the radicals) (or “drift time”) is sufficient, which depends on the reaction rate between these species and the electric field applied. The range of length (typically 5-10 cm, preferable less than 30 cm), together with the pressure and electric field conditions recommended here (see below) have been found to be suitable to the detection of organic radicals,

2) that the electric field inside the drift chamber is typically between 10 and 100 Td at the operated pressure, while requiring a reasonably low entrance voltage (<5000 V). For instance, for an electric field of 100 Td, a drift chamber of length L =5 cm operated at 10 mbar requires a voltage X of:

100 Td=X(V)/5(cm)×3(mbar)/10(mbar)=>X=100×50/3=1666 V

But for the same field, a drift chamber twice as long, L=10 cm, would require twice the voltage, 3333 V.

The pressure at which the drift chamber is operated should be significantly lower (at least 100 mbar) than the pressure of the chemical systems to be analyzed to ensure efficient sampling, but high enough to maintain the electric field below 100 Td.

For instance, to sample radicals from a system at atmospheric pressure (˜1000 mbar) into a drift chamber of length L=5 cm and using a voltage of 2000 V, the pressure in the drift chamber could be as high as 500 mbar, but in that case, the electric field would be only of 2000×3/(5×500)=2.4 Td, which is very low, probably too low for a good detection. But if the pressure in the drift chamber was only 15 mbar, the electric field would be 80 Td, which is the recommended range for the present application.

Outside the inlet opening in the drift chamber is a soft ionization unit mounted, pressure wise communicating with the drift chamber via its inlet opening, producing the reagent ions from a molecular precursor. The soft ionization unit is thus equipped with an inlet valve connected to a source for the molecular precursor, generally operating near atmospheric pressure and introducing the precursor into the soft ionization unit in a flow of, for example humidified nitrogen gas or an inert gas, such as Helium, and with the ion source ionizing the precursor. A range of ion sources can be used as long as they are operational within 5-500 mbar. This includes, but is not limited to, radioactive sources, hollow cathode sources, glow discharge sources, etc, but the soft ionization unit has to be of a soft type, for example one with a radioactive source of about 1-20 mCi.

The ionization technique chosen for the detection of organic radicals in this invention is proton transfer, where the reagent ions are of the form “AH⁺”, where A is the neutral molecular precursor, for instance water (H₂O), ammonia (NH₃) or an organic molecule.

The drift chamber is also equipped with a sampling inlet, allowing for the external sampling of the gas mixtures containing the radicals of interest (i.e. using active means of controlling the sampling flow so that the operating conditions of the drift chamber can be chosen independently from those of the gas mixture in which the radicals are present). This sampling inlet is isolated from the drift chamber by a sampling valve, controlling the sampling flow and the pressure in the drift chamber. The sampling inlet is situated along the drift chamber length, between the inlet opening and the exit pinhole, preferably closer to the inlet opening than to the exit pinhole, typically at one third of the drift chamber length. If the radicals have to be sampled from a closed system or reactor, the sampling inlet has to be equipped with a sampling tube line. The sampling tube line can be made of a range of materials, including, but not limited to, stainless steel, glass, Teflon, etc. The length L of the sampling tube must be chosen so that the residence time of the radicals analyzed meets two criterions, namely 1) is shorter than the diffusion time to the walls of the tube, and 2) residence time is shorter than the radicals lifetime.

At the exit pinhole, the drift chamber is, as said above, connected to a sensitive mass spectrometer, consisting of a mass filter and an ion detecting unit. The exit pinhole must be so small that the vacuum pumps evacuating the mass spectrometer, can keep it on a considerably lower pressure than exists in the drift chamber. This mass spectrometer can be based on a range of standard technologies used in the art (quadrupole, or time-of-flight for the filter, and electron multiplier for the detector) but, in order to detect the radicals, must have a detection limit for trace gas species of less than 100 pptV, or 10⁹ molec. cm⁻³ at atmospheric pressure.

When operating the invention, the drift chamber is under a slight vacuum, typically between 5 and 200 mbar, but possibly as much as 500 mbar, and the mass spectrometer at 10⁻⁵ mbar or less. The flow of ion precursor, A, produced near atmospheric pressure, is sent through the soft ionization unit (5 to 500 mbar) to produce the reagent ions, AH⁺. These reagent ions are accelerated along the axis of the drift chamber by applying a high positive voltage, typically in the range 500-2000 Volts, between the inlet opening (+) of the drift chamber and the exit pinhole (zero earth) at the entrance to the mass spectrometer. Simultaneously, the radicals of interest, for instance peroxy radicals RO₂s, present in a gas system at a pressure larger than the pressure of the drift region, are externally sampled in a flow through the sampling inlet, or if necessary through a sampling tube line and, into the drift chamber. Inside the drift chamber, this sampling flow intersects with the trajectories of the reagent ions, preferably with an angle about 90 degree, allowing for the chemical ionization between the reagent ions and radicals to take place:

RO₂+AH⁺>RO₂H⁺+A

A radical RO₂ of mass m will thus produce a product ion RO₂H⁺ of mass m+1, which allows to differentiate between the different radicals present in the gas system by their mass.

The products ions, RO₂H⁺ are then also accelerated along the drift chamber and towards the exit pinhole at the entrance to the mass spectrometer by the high voltage difference. Inside of the mass spectrometer, they are separated in mass by the mass filter and detected by the detector.

The detection sensitivity, eg detection limit and signal to noise ratio, which are critical in the detection of trace compounds such as organic radicals, can be increased by optimizing various parameters and conditions in the drift chamber system, such as the total pressure and relative ratio of sampling flow to reagent ions flow, typically chosen between 0.01 to 0.8 in order to optimize the signal to noise ratio.

### DETAILED TECHNICAL DESCRIPTION

The different parts, necessary for the invention are aligned along a centerline 1, namely A) a drift chamber 2 designed for proton transfer ionization at intermediate pressure (5-500 mbar) and low electric field (typically <100 Td), B) an external sampling inlet 3 suitable for the detection of radicals, and C) a sensitive mass spectrometer 4 on which the drift chamber 2 is mounted.

The drift chamber 2, preferably symmetrically placed around the centerline 1 is pressure tight and under vacuum, 5-500 mbar, and has in one end an inlet opening 5 for entering reagent ions 6 and in the other end an exit pin hole 7 for exiting product ions 8 entering into the mass spectrometer 4. Preferably the inlet opening 5 and exit pinhole 7 are situated along the centerline 1. The drift chamber 2 can be made of a range of materials, such as, but not limited to stainless steel, glass, Teflon, Delrin, etc, and is typically between 5 and 50 cm in length, and 1 and 20 cm in diameter.

Outside the inlet opening 5 in the drift chamber 2 is a soft ionization unit 9 mounted, pressure wise communicating with the drift chamber via its inlet opening 5, producing the reagent ions 6 from a molecular precursor 11. The soft ionization unit is thus equipped with an inlet valve 10 connected to a source for the molecular precursor 11, generally operating near atmospheric pressure and introducing the precursor 11 into the soft ionization unit in a flow, for example of humidified nitrogen gas or an inert gas, such as nitrogen, and with the ion source ionizing the precursor. A range of ion sources can be used as long as they are operational between 5 and 500 mbar. This includes, but is not limited to, radioactive sources, hollow cathode sources, glow discharge sources, etc, but the soft ionization unit 9 has to be of a soft type, for example one with a radioactive strip source of 1 to 20 mCi.

The ionization technique chosen for the detection of organic radicals in this invention is proton transfer, where the reagent ions are of the form “AH⁺”, where A is the neutral molecular precursor, for instance water (H₂O), ammonia (NH₃) or an organic molecule.

The drift chamber is also equipped with a sampling inlet 3, allowing for the external sampling of the gas mixtures containing the radicals of interest (i.e. sampling taking place outside of the drift chamber). This sampling inlet 3 is isolated from the drift chamber by a sampling valve 12, controlling the sampling flow and indirect the pressure in the drift chamber 2. The sampling inlet 3 is situated along the drift chambers 2 length, between the inlet opening 5 and the exit pinhole 7, preferably closer to the inlet opening 5 than to the exit pinhole 7, typically at one third of the drift chambers 2 length. If the radicals have to be sampled from a closed system or reactor, the sampling inlet has to be equipped with a sampling tube 13. The sampling tube 13 can be made of a range of materials, including, but not limited to, stainless steel, glass, Teflon, etc. The length L of the sampling tube 13 must be chosen so that the residence time of the radicals analyzed meets two criterions, namely 1) is shorter than the diffusion time to the walls of the sampling tube 13, and 2) residence time is shorter than the radicals lifetime.

At the exit pinhole 7, the drift chamber 2 is, as said above, connected to a sensitive mass spectrometer 4, consisting of a mass filter and an ion detecting unit. The exit pinhole 7 must be so small that the vacuum pumps 14 evacuating the mass spectrometer 4, can keep it on a considerably lower pressure than exists in the drift chamber 2. This mass spectrometer 4 can be based on a range of standard technologies used in the art (quadrupole, or time-of-flight for the filter, and electron multiplier for the detector) but, in order to detect the radicals, must have a detection limit for trace gas species of less than 100 pptV, or 10⁹ molec. cm⁻³ at atmospheric pressure.

When operating the invention, the drift chamber 2 is under a slight vacuum by its vacuum pump 15, typically between 5 and 500 mbar, and the mass spectrometer 4 at 10-5 mbar or less. The flow of ion precursor A 11, produced near atmospheric pressure, is fed into the soft ionization unit 9 (5 and 500 mbar) to produce the reagent ions 6, AH+. These reagent ions 6 are accelerated along the center axis 1 of the drift chamber 2 by applying a high positive voltage, typically around 800 Volts, between the inlet opening 5 (+potential) of the drift chamber 2 and its exit pinhole 7 (earth) at the entrance to the mass spectrometer 4. Simultaneously, the radicals of interest, for instance peroxy radicals RO₂, present in a gas system at a pressure larger than the pressure of the drift chamber 2, are externally sampled in a flow through the sampling inlet 3, or if necessary through a sampling tube 13 and, into the drift chamber 2. Inside the drift chamber 2, this sampling flow 16 intersects with the trajectories of the reagent ions 6 with an angle, preferably about 90 degrees, allowing for the chemical ionization between the reagent ions and radicals to take place:

RO₂+AH⁺→RO₂H⁺+A

A radical RO₂ of mass m will thus produce a product ion RO₂H+ of mass m+1, which allows to differentiate between the different radicals present in the gas system by their mass.

The product ions 8, RO₂H⁺ are then also accelerated along the drift chamber 2 and towards the exit pinhole 7 at the entrance to the mass spectrometer by the high voltage difference. Inside of the mass spectrometer 4, they are separated in mass by the mass filter and detected by the detector.

The detection sensitivity, eg a lower detection limit, and signal to noise ratio, which are critical in the detection of trace compounds such as organic radicals, can be increased by optimizing various parameters and conditions in the drift chamber system, such as the total pressure and relative ratio of sampling flow 16 to reagent ions flow 6, typically chosen between 0.01 to 0.8 in order to optimize the signal to noise ratio of the signal to the mass spectrometer 4. The main field of application of the invention would be the detection of gas-phase organic radicals in ambient air, for research activities on air chemistry, air quality, and medical investigation of the impact of air pollution on human health, as well as for routine monitoring activities in the same fields of indoor and outdoor environment monitoring, health monitoring.

1. A method for detection and chemical speciation of organic radicals, i.e. differentiating between organic radicals with different molecular weights, in natural and artificial gas mixtures, including those near atmospheric or high pressure, based on using different devices in a setup, each with special settings, pressure wise communicating with each other, consisting first of a soft ionization unit (9) producing proton transfer reagent ions AH⁺ (6) from a precursor, secondly of a drift chamber (2) where the reagent ions (6) are accelerated by an electric field and are made to collide with a sampling flow (16) of organic radicals introduced in the drift chamber (2) and thirdly of an attached mass spectrometer (4) with a sensitivity eg. detection limit <100 pptV, wherein the product ions (8) are detected and speciated according to their masses,
characterized in that the gas mixture containing the organic radicals to be analyzed is sampled by external sampling through a sampling inlet (3), and that the drift chamber (2) has an internal absolute pressure in the range of 5-500 mbar and has a driving potential in Volt applied over its length depending on the length and the internal pressure in such a way that the intensity of the electric field is less than 100 Td, Townsend.

2. A method according to claim 1, characterized in using the sampling inlet (3) connected to a sampling tube (13) whose length must be chosen so that the residence time of the organic radicals analyzed meets two criterions, namely that it is shorter than the diffusion time to the walls of the sampling tube (13) and that the residence time is shorter than the organic radicals lifetime.

3. A method according to claim 1, characterized in using the sampling inlet (3) situated along the drift chamber's (2) length, between 20% and 50% of the distance between an inlet opening (5) and an exit pinhole (7), eg on the inlet opening (5) side of that distance.

4. A method according to claim 1, characterized in that the ratio of sampling flow (16) to reagent ions flow (6) is changed between 0.01 to 0.7 in order to find an optimum signal to noise ratio of the signal to the mass spectrometer (4).

5. A drift chamber (2), described in claim 1, designed to be under vacuum, designed to be gas tight connected to a mass spectrometer (4) for detection and chemical speciation of organic radicals, i.e. differentiating between organic radicals with different molecular weights, in natural and artificial gas mixtures, including those near atmospheric or high pressure, where reagent ions (6), created in a soft ionization unit (9), producing proton transfer reagent ions AH+ (6) from a precursor situated in one end of the drift chamber (2), which are accelerated by an electric field and are made to collide with a sampling flow (16) of organic radicals introduced in the drift chamber (2) and in a fraction of a second, by the electric field, transport them through an exit pin hole (7) in the other end, to the mass spectrometer (4) , characterized in that the gas mixture containing the radicals to be analyzed are sampled by a so called external sampling arrangement comprising sampling inlet (3) placed in the wall the drift chamber (2) and on its outside connecting to a sampling tube (13) whose length L and inner radius R have to meet two criterions, namely that R over the sampling s peed V, is shorter than the diffusion time to the wall of the sampling tube (13), e.g. L<3,3×Flow, in cm and cm3/s, and the distance L over V also is shorter than the radicals lifetime after entering the sampling tube (13).

6. A drift chamber (2), disclosed in claim 5, characterized in that the sampling inlet (3) is situated along the drift chamber's (2) length, between the soft ionization unit (9), and the exit pinhole (7).

7. A drift chamber (2), according to claim 6, characterized in that the sampling inlet (3) is situated closer to the soft ionization unit (9) than to the exit pinhole (7), between 0.2 and 0.5 of the drift chamber's (2) length.

8. A method according to claim 1, for detection of organic radicals in large gaseous systems near atmospheric pressure (P>50 mbar), such as the natural atmosphere, ambient air, indoor air, large reactors and chambers (>10 L).

9. A method according to claim 1, for detection of organic radicals in gaseous systems with biological or medical applications, such as exposure studies of living organisms, tissues, of cells to radical or oxidizing mixtures, analysis of breath, transpiration, odours, etc.

10. A method according to claim 1, for detection of organic radicals in industrial gaseous systems, such as the production, processing and use of fuels, chemical industry, food industry, detergent industry, fragrance industry, etc.

11. A method according to claim 1, for detection of organic radicals in combustion or energy production system, such as combustion engines and other engines, combustion of regular fuels, biofuels, biomass.
